(19) United States
(12) Patent Application Publication
(10) Pub. No.: US 2022/0257297 A1
(43) Pub. Date: Aug. 18, 2022

(54) PULSE SEQUENCE FOR CARDIAC ABLATION BY IRREVERSIBLE ELECTROPORATION WITH LOW SKELETAL MUSCLE STIMULATION

(71) Applicant: Boston Scientific Scimed Inc, Maple Grove, MN (US)

(72) Inventors: Brendan E. Koop, Ham Lake, MN (US); Allan C. Shuros, St. Paul, MN (US); Jonathan T. Gorzycki, Blaine, MN (US)

(21) Appl. No.: 17/668,908

(22) Filed: Feb. 10, 2022

Related U.S. Application Data
(60) Provisional application No. 63/149,114, filed on Feb. 12, 2021.

(51) Int. Cl. A61B 18/00; A61M 25/00

(52) U.S. Cl. CPC A61B 18/00; A61B 2018/00613; A61B 2018/00577; A61M 25/0082

(57) ABSTRACT

An electroporation ablation system for treating targeted tissue in a patient. The electroporation ablation system including an ablation catheter and an electroporation generator. The ablation catheter including a handle, a shaft having a distal end, and catheter electrodes situated at the distal end of the shaft and spatially arranged to generate electric fields in the targeted tissue in response to electrical pulses. The electroporation generator operatively coupled to the catheter electrodes and configured to deliver the electrical pulses in an electroporation pulse sequence to one or more catheter electrodes. Wherein, the electroporation pulse sequence includes multiple pulse bursts, and each of the multiple pulse bursts includes pulses separated by an inter-pulse length of between 200 and 350 microseconds to reduce muscle stimulation while creating electroporation lesions.

### CROSS REFERENCE TO RELATED APPLICATION

This application claims priority to Provisional Application No. 63/149,114, filed Feb. 12, 2021, which is herein incorporated by reference in its entirety.

### TECHNICAL FIELD

The present disclosure relates to medical apparatus, systems, and methods for ablating tissue in a patient. More specifically, the present disclosure relates to medical apparatus, systems, and methods for ablation of tissue by electroporation.

### BACKGROUND

Ablation procedures are used to treat many different conditions in patients. Ablation may be used to treat cardiac arrhythmias, benign tumors, cancerous tumors, and to control bleeding during surgery. Usually, ablation is accomplished through thermal ablation techniques including radio-frequency (RF) ablation and cryoablation. In RF ablation, a probe is inserted into the patient and radio frequency waves are transmitted through the probe to the surrounding tissue. The radio frequency waves generate heat, which destroys surrounding tissue and cauterizes blood vessels. In cryoablation, a hollow needle or cryoprobe is inserted into the patient and cold, thermally conductive fluid is circulated through the probe to freeze and kill the surrounding tissue. RF ablation and cryoablation techniques indiscriminately kill tissue through cell necrosis, which may damage or kill otherwise healthy tissue, such as tissue in the esophagus, phrenic nerve cells, and tissue in the coronary arteries.

Another ablation technique uses electroporation. In electroporation, or electro-permeabilization, an electric field is applied to cells to increase the permeability of the cell membrane. The electroporation may be reversible or irreversible, depending on the strength of the electric field. If the electroporation is reversible, the increased permeability of the cell membrane may be used to introduce chemicals, drugs, and/or deoxyribonucleic acid (DNA) into the cell, prior to the cell healing and recovering. If the electroporation is irreversible, the affected cells are killed through apoptosis.

Irreversible electroporation (IRE) employs trains of short, high voltage pulses to generate electric fields that are strong enough to kill cells through apoptosis. In ablation of cardiac tissue, IRE may be a safe and effective alternative to the indiscriminate killing of thermal ablation techniques, such as RF ablation and cryoablation. IRE may be used to kill targeted tissue, such as myocardium tissue, by using an electric field strength and duration that kills the targeted tissue but does not permanently damage other cells or tissue, such as non-targeted myocardium tissue, red blood cells, vascular smooth muscle tissue, endothelium tissue, and nerve cells.

In some IRE procedures, the electroporation electrical pulses cause the unwanted side effect of skeletal muscle stimulation (SMS) and engagement. A way of delivering effective IRE energies while avoiding SMS is needed.

### SUMMARY

In Example 1, an electroporation ablation system for treating targeted tissue in a patient. The electroporation ablation system including an ablation catheter and an electroporation generator. The ablation catheter including a handle, a shaft having a distal end, and catheter electrodes situated at the distal end of the shaft and spatially arranged to generate electric fields in the targeted tissue in response to electrical pulses. The electroporation generator operatively coupled to the catheter electrodes and configured to deliver the electrical pulses in an electroporation pulse sequence to one or more catheter electrodes. Wherein, the electroporation pulse sequence includes multiple pulse bursts, and each of the multiple pulse bursts includes pulses separated by an inter-pulse length of between 200 and 350 microseconds to reduce muscle stimulation while creating electroporation lesions.

In Example 2, the system of Example 1, wherein each of the pulses is a biphasic pulse including a positive pulse portion and a negative pulse portion.

In Example 3, the system of Example 2, wherein each of the positive pulse portion and the negative pulse portion has a pulse width of between 1 and 5 microseconds and the biphasic pulse has an inter-phase delay between the positive pulse portion and the negative pulse portion of between 0 and 10 microseconds.

In Example 4, the system of any one of Examples 2 and 3, wherein the positive pulse portion has a positive pulse amplitude as measured from a reference line of between +500 and +2500 and the negative pulse portion has a negative pulse amplitude as measured from the reference line of between −500 and −2500 volts.

In Example 5, the system of any one of Examples 1-4, wherein the multiple pulse bursts are applied to the patient across multiple heart beats.

In Example 6, the system of any one of Examples 1-5, wherein the multiple pulse bursts are applied to the patient across multiple heart beats, one pulse burst per heartbeat.

In Example 7, the system of any one of Examples 1-6, wherein each pulse burst of the multiple pulse bursts is gated to an R-wave in a heartbeat and applied during one or more of a refractory time of the heartbeat, less than 330 milliseconds, and in a 100-250 millisecond window.

In Example 8, the system of any one of Examples 1-7, wherein the electroporation pulse sequence includes at least 50 pulses.

In Example 9, the system of any one of Examples 1-8, wherein the multiple pulse bursts include at least five pulse bursts and each of the pulse bursts includes at least 10 pulses.

In Example 10, an electroporation ablation system for treating targeted tissue in a patient. The electroporation ablation system including an ablation catheter and an electroporation generator. The ablation catheter including a handle, a shaft having a distal end, and catheter electrodes situated at the distal end of the shaft and spatially arranged to generate electric fields in the targeted tissue in response to electrical pulses. The electroporation generator operatively coupled to the catheter electrodes and configured to deliver the electrical pulses in an electroporation pulse sequence to one or more catheter electrodes. Wherein, the electroporation pulse sequence includes multiple pulse bursts applied across multiple heart beats, one pulse burst per heartbeat, each of the multiple pulse bursts including biphasic pulses separated by an inter-pulse length of between 200 and 350 microseconds to provide reduce muscle stimulation while creating irreversible electroporation lesions.

In Example 11, the system of Example 10, wherein each pulse burst of the multiple pulse bursts is gated to an R-wave in the heartbeat and applied during a ventricle refractory period of the heartbeat.

In Example 12, the system of any one of Examples 10 and 11, wherein each of the biphasic pulses includes a positive pulse portion and a negative pulse portion with an inter-phase delay between the positive pulse portion and the negative pulse portion of between 0 and 10 microseconds, and each of the positive pulse portion and the negative pulse portion has a pulse width of between 1 and 5 microseconds.

In Example 13, a method of ablating targeted tissue in a patient by irreversible electroporation. The method including delivering an irreversible electroporation pulse sequence including delivering multiple pulse bursts across multiple heart beats, each of the multiple pulse bursts including biphasic pulses separated by an inter-pulse length of between 200 and 350 microseconds to reduce muscle stimulation while creating irreversible electroporation lesions.

In Example 14, the method of Example 13, wherein delivering multiple pulse bursts across multiple heart beats includes delivering the biphasic pulses that each have a positive pulse portion and a negative pulse portion each having a pulse width of between 1 and 5 microseconds.

In Example 15, the method of Example 13, wherein delivering multiple pulse bursts across multiple heart beats includes delivering the biphasic pulses that each have a positive pulse portion and a negative pulse portion separated by an inter-phase delay of between 0 and 10 microseconds.

In Example 16, an electroporation ablation system for treating targeted tissue in a patient. The electroporation ablation system including an ablation catheter and an electroporation generator. The ablation catheter including a handle, a shaft having a distal end, and catheter electrodes situated at the distal end of the shaft and spatially arranged to generate electric fields in the targeted tissue in response to electrical pulses. The electroporation generator operatively coupled to the catheter electrodes and configured to deliver the electrical pulses in an electroporation pulse sequence to one or more catheter electrodes. Wherein, the electroporation pulse sequence includes multiple pulse bursts, and each of the multiple pulse bursts includes pulses separated by an inter-pulse length of between 200 and 350 microseconds to reduce muscle stimulation while creating electroporation lesions.

In Example 17, the system of Example 16, wherein each of the pulses is a biphasic pulse including a positive pulse portion and a negative pulse portion.

In Example 18, the system of Example 17, wherein each of the positive pulse portion and the negative pulse portion has a pulse width of between 1 and 5 microseconds and the biphasic pulse has an inter-phase delay between the positive pulse portion and the negative pulse portion of between 0 and 10 microseconds.

In Example 19, the system of Example 17, wherein the positive pulse portion has a positive pulse amplitude as measured from a reference line of between +500 and +2500 and the negative pulse portion has a negative pulse amplitude as measured from the reference line of between −500 and −2500 volts.

In Example 20, the system of Example 16, wherein the multiple pulse bursts are applied to the patient across multiple heart beats.

In Example 21, the system of Example 16, wherein the multiple pulse bursts are applied to the patient across multiple heart beats, one pulse burst per heartbeat.

In Example 22, the system of Example 16, wherein each pulse burst of the multiple pulse bursts is gated to an R-wave in a heartbeat and applied during one or more of a refractory time of the heartbeat, less than 330 milliseconds, and in a 100-250 millisecond window.

In Example 23, the system of Example 16, wherein the electroporation pulse sequence includes at least 50 pulses.

In Example 24, the system of Example 16, wherein the multiple pulse bursts include at least five pulse bursts.

In Example 25, the system of Example 16, wherein each of the pulse bursts includes at least 10 pulses.

In Example 26, the system of Example 16, wherein the electroporation pulse sequence is an irreversible electroporation pulse sequence.

In Example 27, the system of Example 16, comprising a surface patch electrode attached to the patient and configured to generate electric fields in the patient in response to the electrical pulses.

In Example 28, the electroporation ablation system for treating targeted tissue in a patient. The electroporation ablation system an ablation catheter and an electroporation generator. The ablation catheter including a handle, a shaft having a distal end, and catheter electrodes situated at the distal end of the shaft and spatially arranged to generate electric fields in the targeted tissue in response to electrical pulses. The electroporation generator operatively coupled to the catheter electrodes and configured to deliver the electrical pulses in an electroporation pulse sequence to one or more catheter electrodes. Wherein, the electroporation pulse sequence includes multiple pulse bursts applied across multiple heart beats, one pulse burst per heartbeat, each of the multiple pulse bursts including biphasic pulses separated by an inter-pulse length of between 200 and 350 microseconds to provide reduce muscle stimulation while creating irreversible electroporation lesions.

In Example 29, the system of Example 28, wherein each pulse burst of the multiple pulse bursts is gated to an R-wave in the heartbeat and applied during a ventricle refractory period of the heartbeat.

In Example 30, the system of Example 28, wherein each of the biphasic pulses includes a positive pulse portion and a negative pulse portion, and each of the positive pulse portion and the negative pulse portion has a pulse width of between 1 and 5 microseconds.

In Example 31, the system of Example 28, wherein each of the biphasic pulses includes a positive pulse portion and a negative pulse portion with an inter-phase delay between the positive pulse portion and the negative pulse portion of between 0 and 10 microseconds.

In Example 32, a method of ablating targeted tissue in a patient by irreversible electroporation. The method including delivering an irreversible electroporation pulse sequence including delivering multiple pulse bursts across multiple heart beats, each of the multiple pulse bursts including biphasic pulses separated by an inter-pulse length of between 200 and 350 microseconds to reduce muscle stimulation while creating irreversible electroporation lesions.

In Example 33, the method of Example 32, wherein delivering multiple pulse bursts across multiple heart beats includes delivering the biphasic pulses that each have a positive pulse portion and a negative pulse portion each having a pulse width of between 1 and 5 microseconds.

In Example 34, the method of Example 32, wherein delivering multiple pulse bursts across multiple heart beats includes delivering the biphasic pulses that each have a positive pulse portion and a negative pulse portion separated by an inter-phase delay of between 0 and 10 microseconds.

In Example 35, the method of Example 32, wherein delivering multiple pulse bursts across multiple heart beats includes delivering one pulse burst per heartbeat.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient and for treating a heart of the patient, using an electrophysiology system, in accordance with embodiments of the subject matter of the disclosure.

FIG. 2A is a diagram illustrating a distal end of a shaft included in a catheter and interactions between electrode pairs, in accordance with embodiments of the subject matter of the disclosure.

FIG. 2B is a diagram illustrating axial electric fields generated by interactions between electrode pairs, in accordance with embodiments of the subject matter of the disclosure.

FIG. 2C is a diagram illustrating circumferential electric fields generated by interactions between electrode pairs in the catheter, in accordance with embodiments of the subject matter of the disclosure.

FIG. 3 is a diagram illustrating a pulse burst portion of a pulse burst generated by the electroporation generator, in accordance with embodiments of the subject matter of the disclosure.

FIG. 4 is a diagram illustrating a graph showing an effective, durable lesion region and a little or no skeletal muscle stimulation region, in accordance with embodiments of the subject matter of the disclosure.

FIG. 5 is a diagram illustrating a graph of the dependence of the little or no skeletal muscle stimulation region on the inter-pulse length, in accordance with embodiments of the subject matter of the disclosure.

FIG. 6 is a diagram illustrating a graph of acceleration, representing skeletal muscle stimulation, versus the number of pulses in a pulse burst, in accordance with embodiments of the subject matter of the disclosure.

FIG. 7 is a diagram illustrating an electroporation pulse sequence that limits or reduces skeletal muscle stimulation while creating effective, durable electroporation lesions, in accordance with embodiments of the subject matter of the disclosure.

FIG. 8 is a diagram illustrating a graph that shows limited or reduced skeletal muscle stimulation while achieving effective and durable lesions, in accordance with embodiments of the subject matter of the disclosure.

FIG. 9 is a diagram illustrating a method of ablating targeted tissue in a patient by irreversible electroporation, in accordance with embodiments of the subject matter of the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

The following detailed description is exemplary in nature and is not intended to limit the scope, applicability, or configuration of the disclosure in any way. Rather, the following description provides some practical illustrations for implementing exemplary embodiments of the present disclosure. Examples of constructions, materials, and/or dimensions are provided for selected elements. Those skilled in the art will recognize that many of the noted examples have a variety of suitable alternatives.

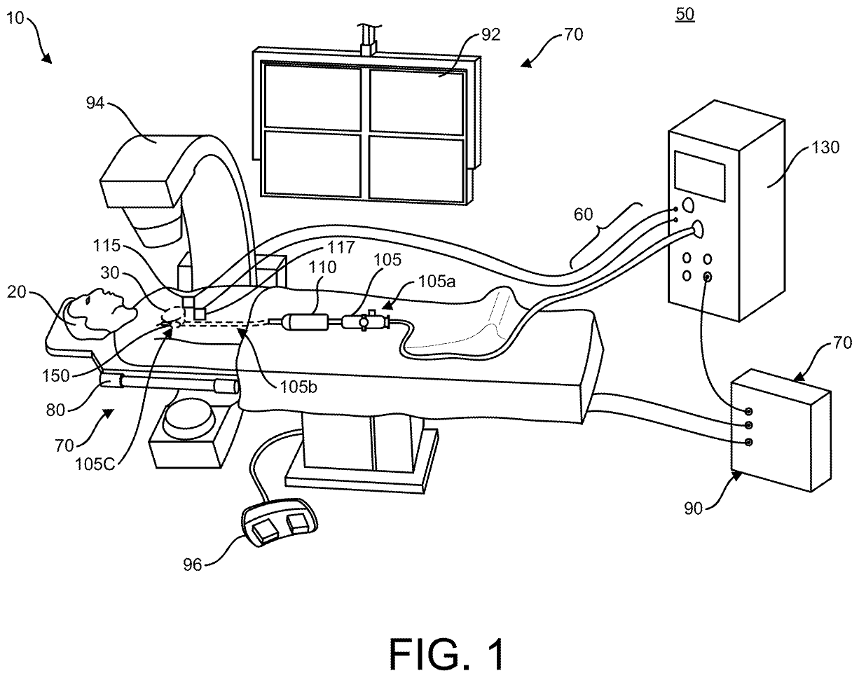

FIG. 1 is a diagram illustrating an exemplary clinical setting 10 for treating a patient 20, and for treating a heart 30 of the patient 20, using an electrophysiology system 50, in accordance with embodiments of the subject matter of the disclosure. The electrophysiology system 50 includes an electroporation system 60 and an electro-anatomical mapping (EAM) system 70, which includes a localization field generator 80, a mapping and navigation controller 90, and a display 92. Also, the clinical setting 10 includes additional equipment such as imaging equipment 94 (represented by the C-arm) and various controller elements, such as a foot controller 96, configured to allow an operator to control various aspects of the electrophysiology system 50. As will be appreciated by the skilled artisan, the clinical setting 10 may have other components and arrangements of components that are not shown in FIG. 1.

The electroporation system 60 includes an electroporation catheter 105, an introducer sheath 110, a surface patch electrode 115, and an electroporation generator 130. Also, in embodiments, the electroporation system 60 includes an accelerometer 117, where the accelerometer 117 can be a separate sensor or part of the surface electrode patch 115. Additionally, the electroporation system 60 includes various connecting elements (e.g., cables, umbilicals, and the like) that operate to functionally connect the components of the electroporation system 60 to one another and to the components of the EAM system 70. This arrangement of connecting elements is not of critical importance to the present disclosure, and one skilled in the art will recognize that the various components described herein may be interconnected in a variety of ways.

In embodiments, the electroporation system 60 is configured to deliver electric field energy to targeted tissue in the patient's heart 30 to create tissue apoptosis, rendering the tissue incapable of conducting electrical signals. The electroporation generator 130 is configured to control functional aspects of the electroporation system 60. In embodiments, the electroporation generator 130 is operable as a pulse generator for generating and supplying pulse sequences to the electroporation catheter 105 and, in some embodiments, the surface patch electrode 115, as described in greater detail herein. In embodiments, the electroporation generator 130 is operable as a pulse generator for generating and supplying pulse sequences to electrodes of the electroporation catheter 105, where electrical energy is supplied as bi-polar pulses, i.e., between two or more electrodes of the electroporation catheter 105. In embodiments, the electroporation generator 130 is operable as a pulse generator for generating and supplying pulse sequences to at least one electrode of the electroporation catheter 105 and the surface patch electrode 115, where the electrical energy is supplied as monopolar pulses, i.e., between the at least one electrode of the electroporation catheter 105 and the surface patch electrode 115. In embodiments, the electroporation generator 130 is operable to receive sensed signals from the accelerometer 117 and based on the received sensed signals act as a pulse generator for generating and supplying pulse sequences to the electroporation catheter 105 and, in some embodiments, the surface patch electrode 115.

In embodiments, the electroporation generator 130 includes one or more controllers, microprocessors, and/or computers that execute code out of memory to control and/or perform the functional aspects of the electroporation catheter system 60. In embodiments, the memory may be part of the one or more controllers, microprocessors, and/or computers, and/or part of memory capacity accessible through a network, such as the world wide web.

In embodiments, the introducer sheath 110 is operable to provide a delivery conduit through which the electroporation catheter 105 may be deployed to the specific target sites within the patient's heart 30. It will be appreciated, however, that the introducer sheath 110 is illustrated and described herein to provide context to the overall electrophysiology system 50, but it is not critical to the novel aspects of the various embodiments described herein.

The EAM system 70 is operable to track the location of the various functional components of the electroporation system 60, and to generate high-fidelity three-dimensional anatomical and electro-anatomical maps of the cardiac chambers of interest. In embodiments, the EAM system 70 may be the RHYTHMIA™ HDx mapping system marketed by Boston Scientific Corporation. Also, in embodiments, the mapping and navigation controller 90 of the EAM system 70 includes one or more controllers, microprocessors, and/or computers that execute code out of memory to control and/or perform functional aspects of the EAM system 70, where the memory, in embodiments, may be part of the one or more controllers, microprocessors, and/or computers, and/or part of memory capacity accessible through a network, such as the world wide web.

As will be appreciated by the skilled artisan, the depiction of the electrophysiology system 50 shown in FIG. 1 is intended to provide a general overview of the various components of the system 50 and is not in any way intended to imply that the disclosure is limited to any set of components or arrangement of the components. For example, the skilled artisan will readily recognize that additional hardware components, e.g., breakout boxes, workstations, and the like, may and likely will be included in the electrophysiology system 50.

The EAM system 70 generates a localization field, via the field generator 80, to define a localization volume about the heart 30, and one or more location sensors or sensing elements on the tracked device(s), e.g., the electroporation catheter 105, generate an output that may be processed by the mapping and navigation controller 90 to track the location of the sensor, and consequently, the corresponding device, within the localization volume. In the illustrated embodiment, the device tracking is accomplished using magnetic tracking techniques, whereby the field generator 80 is a magnetic field generator that generates a magnetic field defining the localization volume, and the location sensors on the tracked devices are magnetic field sensors.

In other embodiments, impedance tracking methodologies may be employed to track the locations of the various devices. In such embodiments, the localization field is an electric field generated, for example, by an external field generator arrangement, e.g., surface electrodes, by intra-body or intra-cardiac devices, e.g., an intracardiac catheter, or both. In these embodiments, the location sensing elements may constitute electrodes on the tracked devices that generate outputs received and processed by the mapping and navigation controller 90 to track the location of the various location sensing electrodes within the localization volume.

In embodiments, the EAM system 70 is equipped for both magnetic and impedance tracking capabilities. In such embodiments, impedance tracking accuracy can, in some instances, be enhanced by first creating a map of the electric field induced by the electric field generator within the cardiac chamber of interest using a probe equipped with a magnetic location sensor, as is possible using the aforementioned RHYTHMIA HDx™ mapping system. One exemplary probe is the INTELLAMAP ORION™ mapping catheter marketed by Boston Scientific Corporation.

Regardless of the tracking methodology employed, the EAM system 70 utilizes the location information for the various tracked devices, along with cardiac electrical activity acquired by, for example, the electroporation catheter 105 or another catheter or probe equipped with sensing electrodes, to generate, and display via the display 92, detailed three-dimensional geometric anatomical maps or representations of the cardiac chambers as well as electro-anatomical maps in which cardiac electrical activity of interest is superimposed on the geometric anatomical maps. Furthermore, the EAM system 70 may generate a graphical representation of the various tracked devices within the geometric anatomical map and/or the electro-anatomical map.

While the EAM system 70 is shown in combination with the electroporation system 60 to provide a comprehensive depiction of an exemplary clinical setting 10, the EAM system 70 is not critical to the operation and functionality of the electroporation system 60. That is, in embodiments, the electroporation system 60 can be employed independently of the EAM system 70 or any comparable electro-anatomical mapping system.

In the illustrated embodiment, the electroporation catheter 105 includes a handle 105a, a shaft 105b, and an electroporation electrode arrangement 150, which is described further hereinafter. The handle 105a is configured to be operated by a user to position the electroporation electrode arrangement 150 at the desired anatomical location. The shaft 105b has a distal end 105c and generally defines a longitudinal axis of the electroporation catheter 105. As shown, the electroporation electrode arrangement 150 is located at or proximate the distal end 105c of the shaft 105b. In embodiments, the electroporation electrode arrangement 150 is electrically coupled to the electroporation generator 130, to receive electrical pulse sequences or pulse trains, thereby selectively generating electrical fields for ablating the target tissue by irreversible electroporation.

In embodiments, the surface patch electrode 115 includes a conductive electrode that can be attached to the body of the patient 20, such as to the thorax of the patient. The surface patch electrode 115, including the conductive electrode, is electrically coupled to the electroporation generator 130 to act as a return path or sink for electrical energy in the system and to receive electrical pulse sequences or pulse trains from the electroporation generator 130, thereby acting as a source for electrical energy and selectively generating electrical fields for ablating the target tissue by irreversible electroporation. In embodiments, the surface patch electrode 115 acts as a return or sink for electrical energy received by the electroporation catheter 105 and the electroporation electrode arrangement 150. In embodiments, the surface patch electrode 115 acts as a source for electrical energy and the electroporation catheter 105 including the electroporation electrode arrangement 150 acts as the return or sink for the sourced electrical energy.

In embodiments, the electroporation system 60 includes the accelerometer 117 that may be attached to the body of the patient 20, such as to the thorax of the patient, and electrically coupled to the electroporation generator 130. The accelerometer 117 is configured to sense contraction of the skeletal muscle system of the patient. The signals from the accelerometer 117 are received by the electroporation generator 130, which processes the signals to determine whether the skeletal muscle system of the patient is contracting.

Also, in embodiments, the local impedance of the target tissue and tissue surrounding the target tissue can be measured to calculate pre-ablation and post-ablation values for evaluation of the lesion efficacy.

The electroporation system 60 is operable to generate an electroporation pulse sequence that includes multiple pulse bursts, where each of the multiple pulse bursts includes multiple pulses. The electroporation generator 130 is operatively coupled to catheter electrodes of the electroporation catheter 105 and configured to deliver the electrical pulses in the electroporation pulse sequence to one or more of the catheter electrodes and/or the surface patch electrode 115. The electroporation pulse sequence is configured to reduce muscle stimulation while creating electroporation lesions. In embodiments, the electroporation pulse sequence is an IRE pulse sequence configured to ablate targeted tissue. In embodiments, the electroporation pulse sequence is a series of electroporation pulses configured to cause irreversible damage to the targeted tissue.

Each of the multiple pulse bursts includes pulses separated by an inter-pulse length or delay between pulses. In embodiments, each of the pulses is a biphasic pulse including a positive pulse portion and a negative pulse portion and, in embodiments, the inter-pulse length is between 200 and 350 microseconds to reduce muscle stimulation while creating electroporation lesions. In some embodiments, the multiple pulse bursts are applied to the patient across multiple heart beats and, in some embodiments, one pulse burst of the multiple pulse bursts is applied per heartbeat.

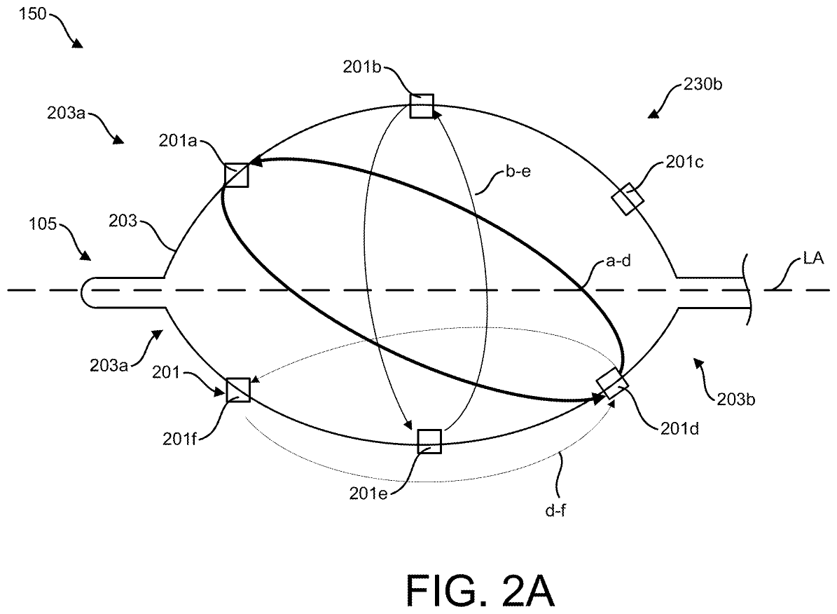

FIGS. 2A-2C show features of the electroporation catheter 105 that includes the electroporation electrode arrangement 150 according to exemplary embodiments. In the illustrated embodiment in FIG. 2A, the electroporation electrode arrangement 150 includes a plurality of electrodes 201a, 201b, 201c, 201d, 201e, and 201f arranged in a three-dimensional electrode array, such that respective ones of the electrodes 201a, 201b, 201c, 201d, 201e, and 201f are spaced from one another axially (i.e., in the direction of the longitudinal axis LA), circumferentially about the longitudinal axis LA and/or radially relative to the longitudinal axis LA. In some embodiments, the electrodes 201a, 201b, 201c, 201d, 201e, and 201f are each individually, selectively addressable via the electroporation generator 130 (FIG. 1) to define a plurality of anode-cathode electrode pairs, each capable of receiving an electrical pulse sequence from the electroporation generator 130 and, consequently, creating an electric field capable of selectively targeting tissue via electroporation, including ablating target tissue via IRE. FIG. 2A schematically illustrates interactions (e.g., current flows forming electric fields) between electrode pairs formed between electrodes 201 (e.g., 201a, 201b, 201c, 201d, 201e, and 201f) included in the electroporation catheter 105. In this figure, interactions are shown as paired arrows (e.g., a-d, b-e, and d-f) indicating current flows between electrodes 201. And electrode pairs (e.g., 201a and 201d, 201b and 201e, and 201d and 201f) are shown with their respective current flows (e.g., a-d, b-e, and d-f) labeled.

FIG. 2B is a diagram illustrating electric fields 210 generated by interactions between electrode pairs in the electroporation catheter 105. In this figure, axially oriented electric fields 210 are shown positioned at an ostium 221 between the left atrium 223 and the left inferior pulmonary vein 225. In embodiments, the axially oriented electric fields 210 are produced by delivering electrical pulses to axially spaced anodes and cathodes.

FIG. 2C is also a diagram illustrating electric fields 210 generated by interactions between electrode pairs in the electroporation catheter 105. But here, the electric fields 210 are circumferentially oriented. In embodiments, the circumferentially oriented electric fields 210 are produced by delivering electrical pulses to circumferentially spaced anodes (“A”) and cathodes (“C”).

FIGS. 2A-2C show that multiple electric fields 210 may be generated simultaneously and/or sequentially and in axial and circumferential orientations. For example, in embodiments, axially and circumferentially oriented electric fields 210 can be generated non-simultaneously in a pre-defined sequence by selectively controlling the timing of the delivery of the electric pulses to the respective electrodes 201. In addition, it is understood that intermittently generated electric fields 210 caused by staggered interactions between sets of electrode pairs and electric field orientations other than axial and circumferential are not beyond the scope of this disclosure.

As may be seen in FIG. 2A, the electroporation electrode arrangement 150 may include a plurality of individually addressable electrodes 201 (e.g., anodes or cathodes) arranged to selectively define a plurality of electrode pairs (e.g., anode-cathode pairs). Each anode-cathode pair may be configured to generate an electric field when a pulse sequence is delivered thereto. The plurality of anode-cathode pairs may include at least two of a first anode-cathode pair, a second anode-cathode pair, and a third anode-cathode pair. The first anode-cathode pair may be arranged to generate a first electric field oriented generally circumferentially relative to the longitudinal axis when a first pulse sequence is delivered thereto. The second anode-cathode pair may be arranged to generate a second electric field oriented generally in a same direction as the longitudinal axis when a second pulse sequence is delivered thereto. The third anode-cathode pair may be arranged to generate a third electric field oriented generally transverse to the longitudinal axis when a third pulse sequence is delivered thereto. In embodiments, any combination of the first, second, and third pulse sequences may be delivered simultaneously or intermittently and may take a variety of forms.

In embodiments, the electroporation electrode arrangement 150 may be configured to structurally arrange the electrodes 201a, 201b, 201c, 201d, 201e, and 201f into a distally-located first region and a more proximally-located second region. As such, electrode pairs may be formed across various electrodes 201 in the electroporation electrode arrangement 150 between first and second regions. For example, the electrodes 201d and 201f may be configured to form an electrode pair. Similarly, the electrodes 201a and 201d or electrodes 201b and 201e or the combination thereof may be selected to form respective electrode pairs. Thus, the electrode pairs may comprise axially spaced electrodes, transversely spaced electrodes, or circumferentially spaced electrodes. Additionally, in embodiments, a given electrode (e.g., 201d) may serve as a common electrode in at least two electrode pairs to generate electric fields 210.

FIG. 2B shows a diagram of exemplary electric fields 210 that may be generated by the electroporation electrode arrangement 150. The electroporation electrode arrangement 150 may be configured to generate a multidirectional electric field 210 when at least one pulse sequence is delivered thereto. The multidirectional electric field 210 may include at least two of the following directions relative to the longitudinal axis: generally axial, circumferential, and transverse. As used herein, transverse may mean at any non-parallel angle relative to the longitudinal axis. As described, the electroporation electrode arrangement 150 may be configured to be operatively couple to the electroporation generator 130 that is configured to generate at least one electroporation pulse sequence. The electroporation electrode arrangement 150 may be configured to receive the at least one electroporation pulse sequence from the electroporation generator 130. Thus, the electroporation electrode arrangement 150 and the electroporation generator 130 may be in operative communication with each other. In this disclosure, such communication may be used to generate electric fields 210 that are at least substantially gapless.

Undesired gaps in electric fields 210 generated by the electroporation electrode arrangement 150 may be limited or at least substantially eliminated. Such gaps may potentially lead to lesion gaps and therefore require multiple repositions of a catheter, for example. Overlapping electric fields 210 may at least substantially limit the number of such gaps. In embodiments, at least some of the electric fields 210 generated in the first pulse sequence set may overlap at least partially with each other. For example, adjacent electric fields 210 (e.g., axial, transverse, and/or circumferential) in a combined electric field 211 may intersect one another so that there are limited to no gaps in the combined electric field 211. Overlapping may occur at or near the periphery of adjacent electric fields 210 or may occur over a preponderance or majority of one or more adjacent electric fields 210. In this disclosure, adjacent means neighboring electrodes 201 or electrodes 201 otherwise near each other. The electroporation generator may be configured to generate pulse sequences used in generating overlapping electric fields.

The configuration of the electroporation electrode arrangement 150 in the various embodiments may take on any form, whether now known or later developed, suitable for a three-dimensional electrode structure. In exemplary embodiments, the electroporation electrode arrangement 150 may be in the form of a splined basket catheter, with respective electrodes 201a, 201b, 201c, 201d, 201e, and 201f positioned on a plurality of splines in any manner known in the art. In embodiments, the electroporation electrode arrangement 150 can be formed on an expandable balloon, e.g., with electrodes formed on flexible circuit branches or individual traces disposed on the balloon surface. In other embodiments, the electroporation electrode arrangement 150 may be in the form of an expandable mesh. In short, the particular structure used to form the electroporation electrode arrangement 150 is not critical to the embodiments of the present disclosure.

In embodiments, the electroporation system 60 is configured to deliver electric field energy to targeted tissue in the patient's heart 30 to create tissue apoptosis, rendering the tissue incapable of conducting electrical signals, using at least one of the plurality of electrodes 201a, 201b, 201c, 201d, 201e, and 201f of the electroporation electrode arrangement 150 and, in some embodiments, the surface patch electrode 115. In embodiments, the electroporation generator 130 is operable as a pulse generator for generating and supplying pulse sequences to two or more of the plurality of electrodes 201a, 201b, 201c, 201d, 201e, and 201f of the electroporation electrode arrangement 150, where electrical energy is supplied as bi-polar pulses, i.e., between two or more of the plurality of electrodes 201a, 201b, 201c, 201d, 201e, and 201f of the electroporation electrode arrangement 150. In embodiments, the electroporation generator 130 is operable as a pulse generator for generating and supplying pulse sequences to at least one electrode of the plurality of electrodes 201a, 201b, 201c, 201d, 201e, and 201f of the electroporation electrode arrangement 150, where the electrical energy is supplied as monopolar pulses, i.e., between the at least one electrode of the plurality of electrodes 201a, 201b, 201c, 201d, 201e, and 201f of the electroporation electrode arrangement 150 and the surface patch electrode 115.

Achieving effective, durable lesions while avoiding excessive skeletal muscle stimulation is a difficult task that includes optimizing multiple pulse sequence characteristics, such as the number of pulse bursts in a pulse sequence, the number of pulses in a pulse burst, the total number of pulses in the pulse sequence, pulse widths, pulse amplitudes, and spacing between pulses in a pulse burst.

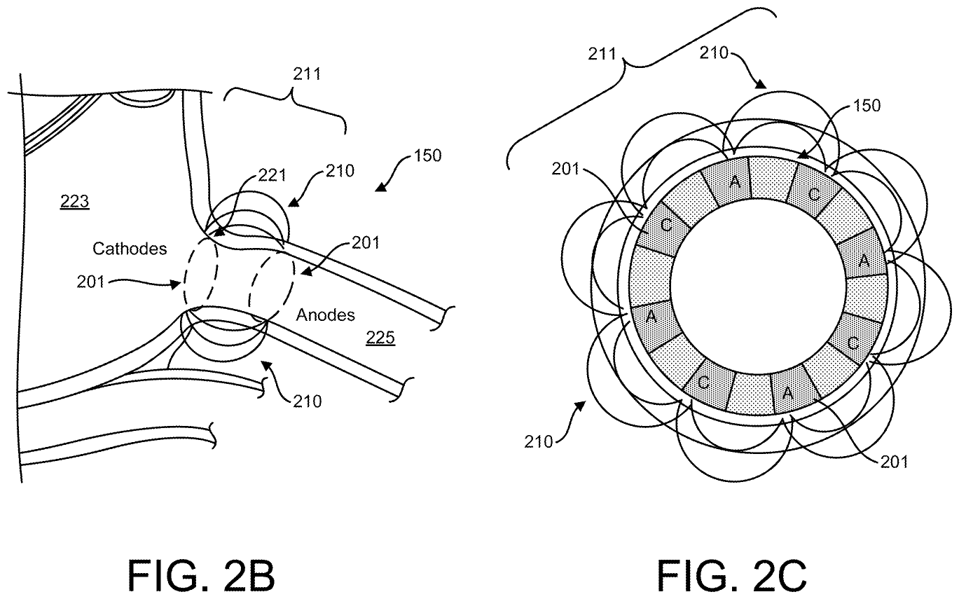

FIG. 3 is a diagram illustrating a pulse burst portion 300 of a pulse burst generated by the electroporation generator 130, in accordance with embodiments of the subject matter of the disclosure. As described, the electroporation system 60 is operable to generate an electroporation pulse sequence that includes multiple pulse bursts, where each of the multiple pulse bursts includes multiple pulses. In embodiments, the electroporation pulse sequence includes at least 5 pulse bursts. In embodiments, one or more of the multiple pulse bursts includes at least 10 pulses, such as at least 10 biphasic pulses. In embodiments, one or more of the multiple pulse bursts includes between 10 and 60 pulses, such as between 10 and 60 biphasic pulses. In some embodiments, the electroporation pulse sequence includes a total of at least 50 pulses.

The pulse burst portion 300 includes three biphasic pulses 302, 304, and 306. Each of the biphasic pulses 302, 304, and 306 includes a positive pulse and a negative pulse, such that biphasic pulse 302 includes positive pulse 302a and negative pulse 302b, biphasic pulse 304 includes positive pulse 304a and negative pulse 304b, and biphasic pulse 306 includes positive pulse 306a and negative pulse 306b.

Each of the biphasic pulses 302, 304, and 306 has pulse characteristics as follows: a positive pulse width (PPW) 308, a negative pulse width (NPW) 310, an inter-phase delay (IPhD) 312, a positive pulse amplitude (PPA) 314, and a negative pulse amplitude (NPA) 316. Also, the pulses, such as pulses 302, 304, and 306, are separated by an inter-pulse length or delay (IPD) 318 between each of the pulses 302, 304 and 306. In embodiments, the inter-pulse length 318 is between 200 and 350 microseconds to reduce muscle stimulation while creating electroporation lesions.

Characteristics, such as pulse widths including the positive pulse width 308 and the negative pulse width 310, pulse amplitudes including the positive pulse amplitude 314 and the negative pulse amplitude 316, and the inter-pulse length 318, are optimized to achieve effective, durable lesions while avoiding excessive skeletal muscle stimulation.

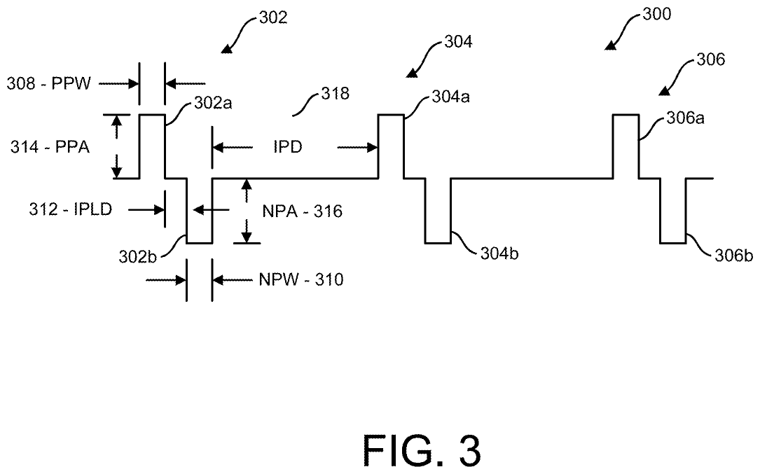

FIG. 4 is a diagram illustrating a graph 400 showing an effective, durable lesion region 402 and a little or no skeletal muscle stimulation region 404, in accordance with embodiments of the subject matter of the disclosure. In the graph 400, the effective, durable lesion region 402 lies above line 406 and the little or no skeletal muscle stimulation region 404 lies below line 408.

The graph 400 is a graph of pulse width 410, such as positive pulse width 308 and negative pulse width 310, along the x-axis and pulse amplitude 412, such as positive pulse amplitude 314 and negative pulse amplitude 316, along the y-axis. In this example, the positive pulse width 308 and the negative pulse width 310 are equal or the same, and the positive pulse amplitude 314 and the negative pulse amplitude 316 are equal or the same.

The design goal 414 for achieving effective durable lesions with little or no skeletal muscle stimulation, lies between the lines 406 and 408, where the effective, durable lesion region 402 overlaps the little or no skeletal muscle stimulation region 404. As illustrated, the design goal 414 is situated where the pulse width 410 is relatively small and the pulse amplitude 412 is relatively large or high.

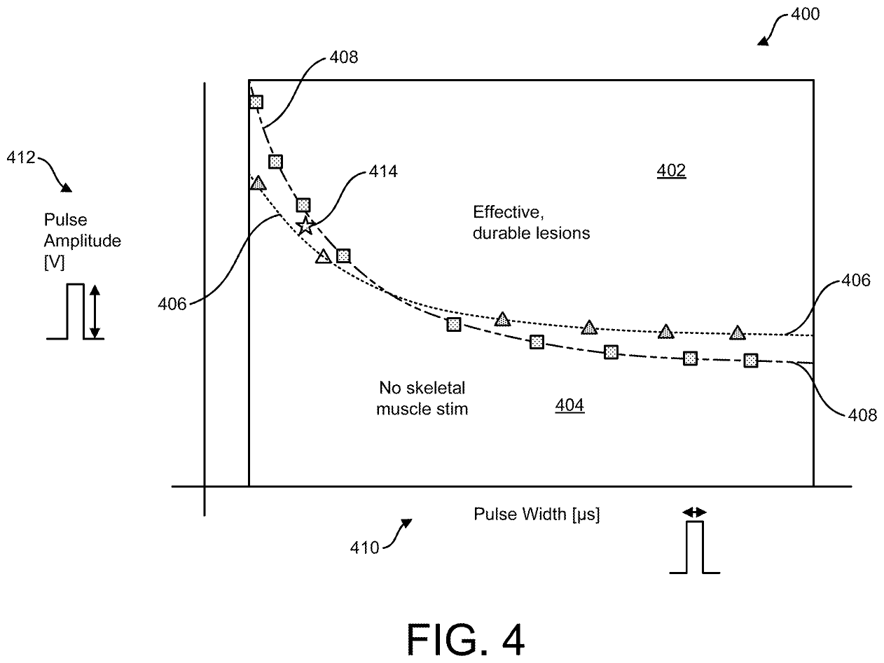

FIG. 5 is a diagram illustrating a graph 500 of the dependence of the little or no skeletal muscle stimulation region 404 on the inter-pulse length 318, in accordance with embodiments of the subject matter of the disclosure. The graph 500 is a graph of pulse width 502, such as positive pulse width 308 and negative pulse width 310, along the x-axis and pulse amplitude 504, such as positive pulse amplitude 314 and negative pulse amplitude 316, along the y-axis. In this example, the positive pulse width 308 and the negative pulse width 310 are equal or the same, and the positive pulse amplitude 314 and the negative pulse amplitude 316 are equal or the same.

In the graph 500, with an inter-pulse length 318 of 2 microseconds, the little or no skeletal muscle stimulation region 404 is situated below line 506, and with an inter-pulse length 318 of 40 microseconds, the little or no skeletal muscle stimulation region 404 is situated below line 508. Thus, increasing the inter-pulse length 318, increases the little or no skeletal muscle stimulation region 404, and increasing the inter-pulse length 318 causes less skeletal muscle stimulation. Also, it has been found that increasing the inter-pulse length 318 has little or no effect on lesion efficacy and may even be beneficial to lesion efficacy.

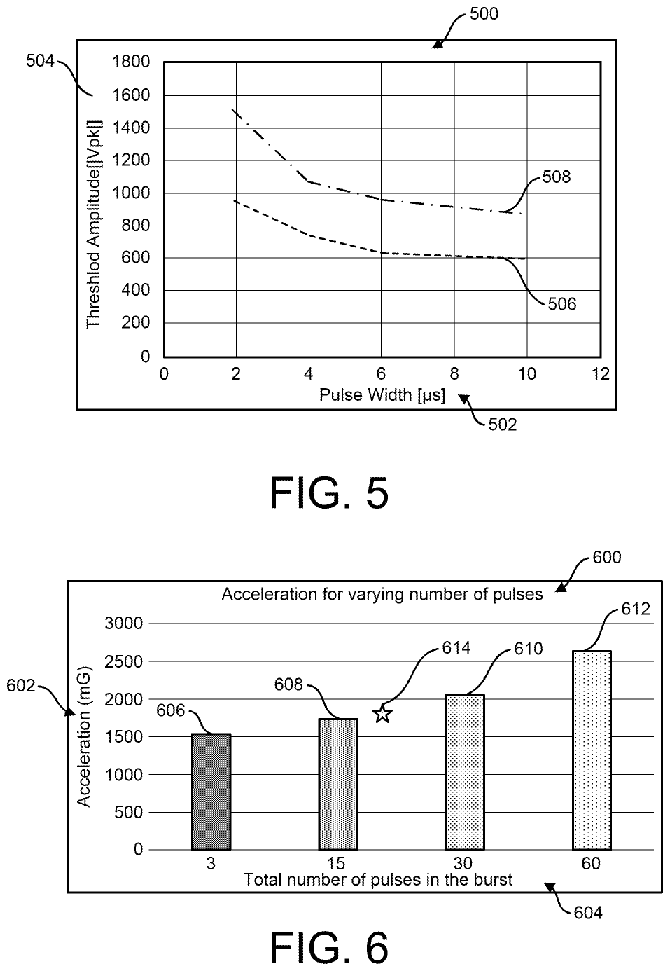

FIG. 6 is a diagram illustrating a graph 600 of acceleration 602, representing skeletal muscle stimulation, versus the number of pulses in a pulse burst 604, in accordance with embodiments of the subject matter of the disclosure. The acceleration 602 is measured in milli-Gs (mG). Also, in this example, acceleration measurements were taken using pulse widths of 6 micro-seconds for each of the positive pulse width 308 and the negative pulse width 310, an inter-phase delay 312 of 2 microseconds, and an inter-pulse length 318 of 40 microseconds.

As illustrated in graph 600, the acceleration was about 1500 mG with 3 pulses in a pulse burst 606, about 1700 mG with 15 pulses in a pulse burst 608, about 2000 mG with 30 pulses in a pulse burst 610, and about 2600 mG with 60 pulses in a pulse burst 612. Thus, skeletal muscle stimulation, as measured by the acceleration 602, increases as the number of pulses in a pulse burst increases.

It has been found that increasing the number of pulses in a pulse burst results in better lesion efficacy and irreversibility, with little benefit accruing after more than 40 pulses in a pulse burst. However, to limit skeletal muscle stimulation, since fewer pulses in a pulse burst results in less skeletal muscle stimulation, in embodiments, 20 pulses per pulse burst 614 was selected as an optimum operating parameter. Also, in some embodiments of an electroporation pulse sequence, 5 pulse bursts with 20 pulses per pulse burst 614 was chosen, for a total of 100 pulses to be applied via the electroporation pulse sequence.

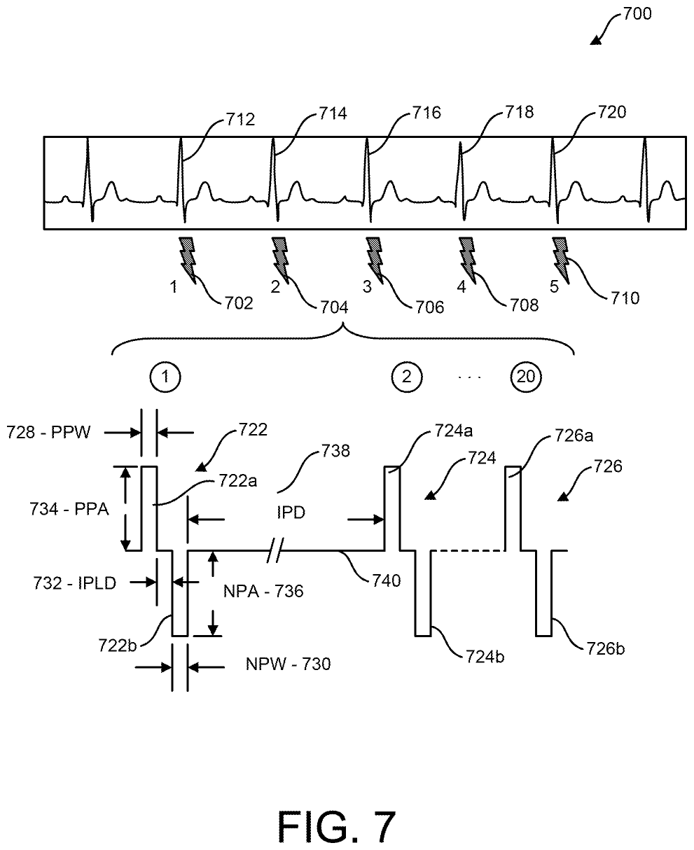

FIG. 7 is a diagram illustrating an electroporation pulse sequence 700 that limits or reduces skeletal muscle stimulation while creating effective, durable electroporation lesions, in accordance with embodiments of the subject matter of the disclosure. In embodiments, the electroporation pulse sequence 700 is an irreversible electroporation pulse sequence.

In the current example, the electroporation pulse sequence 700 includes 5 pulse bursts 702, 704, 706, 708, and 710 to be applied to a patient's heart, one pulse burst per heartbeat 712, 714, 716, 718, and 720, respectively. In embodiments, each of the pulse bursts 702, 704, 706, 708, and 710 is gated to an R-wave in a corresponding one of the heartbeats 712, 714, 716, 718, and 720 and applied during one or more of a refractory time of the heartbeat, less than 330 milliseconds, and in a 100-250 millisecond window.

In other examples and embodiments, the electroporation pulse sequence includes multiple pulse bursts to be applied to the patient's heart, where more than one pulse burst can be applied during a heartbeat and no pulse burst may be applied during a heartbeat. In these examples and embodiments, the pulse bursts are supplied asynchronously during heartbeats with at least a minimum time between pulse bursts.

Also, in these examples and embodiments, the pulses bursts may or may not be gated to an R-wave in a heartbeat.

In some embodiments, in the current example, the electroporation pulse sequence 700 includes more than 5 pulse bursts, such as 10 or 15 or more pulse bursts. Also, in other embodiments, more than one pulse burst can be applied during one heartbeat and, in some embodiments, heartbeats can be skipped, such that one or more pulse bursts is applied to one heartbeat and no pulse bursts are applied to the following heartbeat or heartbeats, until later in the sequence of heartbeats.

In the current example, each of the 5 pulse bursts 702, 704, 706, 708, and 710 includes 20 biphasic pulses. Thus, the electroporation pulse sequence 700 includes 20 biphasic pulses in each of 5 pulse bursts 702, 704, 706, 708, and 710, for a total of 100 biphasic pulses in the electroporation pulse sequence 700. In other embodiments, the electroporation pulse sequence 700 can include at least 10 pulses, such as at least 10 biphasic pulses, in each of the pulse bursts. In some embodiments, the electroporation pulse sequence 700 includes between 10 and 60 pulses, such as between 10 and 60 biphasic pulses, in each of the pulse bursts. Also, in other embodiments, the electroporation pulse sequence 700 includes a total of at least 50 pulses, such as at least 50 biphasic pulses.

By way of example, the first pulse burst 702 includes 20 biphasic pulses, including the illustrated biphasic pulses 722, 724, and 726. Each of the 20 biphasic pulses is similar to biphasic pulses 722, 724, and 726 and includes a positive pulse portion and a negative pulse portion, such that biphasic pulse 722 includes positive pulse 722a and negative pulse 722b, biphasic pulse 724 includes positive pulse 724a and negative pulse 724b, and biphasic pulse 726 includes positive pulse 726a and negative pulse 726b.

The biphasic pulses 722, 724, and 726 have pulse characteristics including a positive pulse width (PPW) 728, a negative pulse width (NPW) 730, an inter-phase delay (IPhD) 732, a positive pulse amplitude (PPA) 734, and a negative pulse amplitude (NPA) 736. Also, the pulses are separated by an inter-pulse length or delay (IPD) 738 between adjacent pulses in the sequence of 20 biphasic pulses.

These characteristics can be and are optimized to achieve effective, durable lesions while avoiding excessive skeletal muscle stimulation. The electrical pulses can be applied via the electrodes 201 of the catheter 105 and/or the surface patch electrode 115.

In one example embodiment, optimized to achieve effective, durable lesions while avoiding excessive skeletal muscle stimulation, each of the positive pulse width (PPW) 728 and the negative pulse width (NPW) 730 has a pulse width of 2 microseconds, the inter-phase delay (IPhD) 732 is 2 microseconds, the positive pulse amplitude (PPA) 734 as measured from reference line 740 is between +500 and +2500 volts, the negative pulse amplitude (NPA) 736 as measured from the reference line 740 is between −500 and −2500 volts, and the inter-pulse length 738 is between 200 and 350 microseconds to limit and reduce muscle stimulation while creating electroporation lesions. In some embodiments, the positive pulse amplitude (PPA) 734 as measured from the reference line 740 is between +1200 and +2500 volts and, in some embodiments, the negative pulse amplitude (NPA) 736 as measured from the reference line 740 is between −1200 and −2500 volts. In some embodiments, the reference line 740 is at 0 volts.

In other embodiments, each of the positive pulse width (PPW) 728 and the negative pulse width (NPW) 730 has a pulse width between 1 and 5 microseconds and, in some embodiments, the inter-phase delay (IPhD) 732 is between 0 and 10 microseconds.

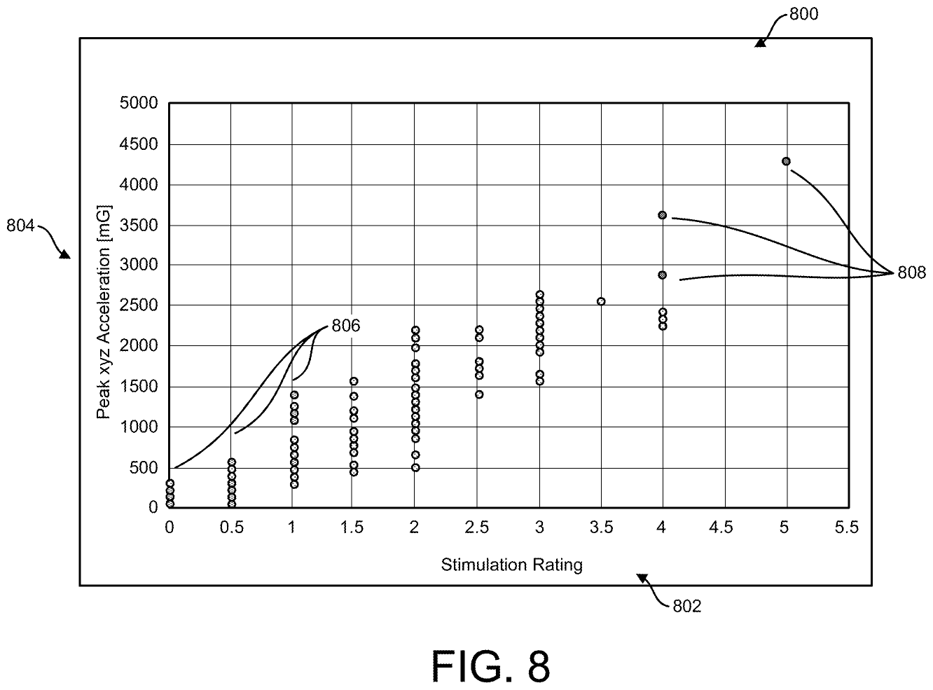

FIG. 8 is a diagram illustrating a graph 800 that shows limited or reduced skeletal muscle stimulation while achieving effective and durable lesions, in accordance with embodiments of the subject matter of the disclosure. The graph 800 displays an observed stimulation rating 802 versus peak xyz acceleration 804 measured in mGs. It is to be understood, that the graph 800 data was gathered in relation to a swine model and as such are directionally applicable to humans as well.

The stimulation rating 802 is chosen based on the following: a zero (0) indicates that no skeletal muscle stimulation is observed; a 1 indicates local palpitations, but nothing gross and no phrenic node stimulation; a 2 indicates visible movement of the torso, with the body shaking; a 3 indicates more violent visible movement of the torso, with the body shaking; and a 4 indicates that delivery of the electroporation pulse sequence looks like a defibrillator shocking the body.

Applying the electroporation pulse sequence described herein and as described in the description of FIG. 7 results in dots indicated at 806, where the stimulation rating 802 is 1 or less and the peak xyz acceleration 804 is below 1500 mGs. This is opposed to the other dots in the graph 800, including the high dosage dots indicated at 808, which are at a stimulation rating 802 of 4 or above and a peak xyz acceleration 804 of about 2800 mGs and above.

Thus, by applying the electroporation pulse sequence as described herein, the electroporation system achieves limiting or reducing the skeletal muscle stimulation while achieving effective, durable electroporation ablation lesions.

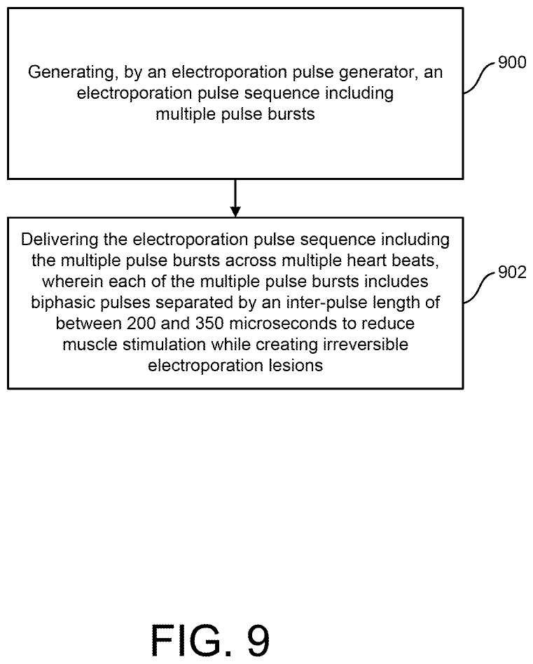

FIG. 9 is a diagram illustrating a method of ablating targeted tissue in a patient by irreversible electroporation, in accordance with embodiments of the subject matter of the disclosure.

At 900, the method includes generating, by an electroporation pulse generator, an electroporation pulse sequence including multiple pulse bursts. In embodiments, the electroporation pulse generator is like the electroporation generator 130.

At 902, the method includes delivering the electroporation pulse sequence including the multiple pulse bursts across multiple heart beats, wherein each of the multiple pulse bursts includes biphasic pulses separated by an inter-pulse length of between 200 and 350 microseconds to reduce muscle stimulation while creating irreversible electroporation lesions.

In embodiments, the method includes delivering multiple pulse bursts across multiple heart beats, one pulse burst per heartbeat. In other embodiments, the method includes delivering more than one pulse burst during one heartbeat and, in some embodiments, the method includes skipping one or more heartbeats, such that one or more pulse bursts is applied to one heartbeat and no pulse bursts are applied to the following heartbeat or heartbeats, until later in the sequence of heartbeats.

Also, in embodiments, the method includes gating each of the pulse bursts to an R-wave in a corresponding one of the heartbeats and, in some embodiments, the method includes applying the pulse burst during one or more of a refractory time of the heartbeat, less than 330 milliseconds, and in a 100-250 millisecond window.

In addition, in embodiments, the method includes delivering biphasic pulses that each have a positive pulse portion and a negative pulse portion, with each having a pulse width of between 1 and 5 microseconds. Also, in embodiments, the method includes delivering biphasic pulses that each have a positive pulse portion and a negative pulse portion separated by an inter-phase delay of between 0 and 10 microseconds. In addition, in some embodiments, the method includes delivering the positive pulse amplitude (PPA) 734 as measured from the reference line 740 between +500 and +2500 volts and, in some embodiments, the method includes delivering the negative pulse amplitude (NPA) 736 as measured from the reference line 740 between −500 and −2500 volts. In some embodiments, the method includes delivering the positive pulse amplitude (PPA) 734 as measured from the reference line 740 between +1200 and +2500 volts and, in some embodiments, the method includes delivering the negative pulse amplitude (NPA) 736 as measured from the reference line 740 between −1200 and −2500 volts. In some embodiments, the reference line 740 is at 0 volts.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

What is claimed is:

1. An electroporation ablation system for treating targeted tissue in a patient, the electroporation ablation system comprising:
an ablation catheter including:
a handle;
a shaft having a distal end; and
catheter electrodes situated at the distal end of the shaft and spatially arranged to generate electric fields in the targeted tissue in response to electrical pulses; and
an electroporation generator operatively coupled to the catheter electrodes and configured to deliver the electrical pulses in an electroporation pulse sequence to one or more catheter electrodes,
wherein the electroporation pulse sequence includes multiple pulse bursts, and each of the multiple pulse bursts includes pulses separated by an inter-pulse length of between 200 and 350 microseconds to reduce muscle stimulation while creating electroporation lesions.

2. The electroporation ablation system of claim 1, wherein each of the pulses is a biphasic pulse including a positive pulse portion and a negative pulse portion.

3. The electroporation ablation system of claim 2, wherein each of the positive pulse portion and the negative pulse portion has a pulse width of between 1 and 5 microseconds and the biphasic pulse has an inter-phase delay between the positive pulse portion and the negative pulse portion of between 0 and 10 microseconds.

4. The electroporation ablation system of claim 2, wherein the positive pulse portion has a positive pulse amplitude as measured from a reference line of between +500 and +2500 and the negative pulse portion has a negative pulse amplitude as measured from the reference line of between −500 and −2500 volts.

5. The electroporation ablation system of claim 1, wherein the multiple pulse bursts are applied to the patient across multiple heart beats.

6. The electroporation ablation system of claim 1, wherein the multiple pulse bursts are applied to the patient across multiple heart beats, one pulse burst per heartbeat.

7. The electroporation ablation system of claim 1, wherein each pulse burst of the multiple pulse bursts is gated to an R-wave in a heartbeat and applied during one or more of a refractory time of the heartbeat, less than 330 milliseconds, and in a 100-250 millisecond window.

8. The electroporation ablation system of claim 1, wherein the electroporation pulse sequence includes at least 50 pulses.

9. The electroporation ablation system of claim 1, wherein the multiple pulse bursts include at least five pulse bursts.

10. The electroporation ablation system of claim 1, wherein each of the pulse bursts includes at least 10 pulses.

11. The electroporation ablation system of claim 1, wherein the electroporation pulse sequence is an irreversible electroporation pulse sequence.

12. The electroporation ablation system of claim 1, comprising a surface patch electrode attached to the patient and configured to generate electric fields in the patient in response to the electrical pulses.

13. An electroporation ablation system for treating targeted tissue in a patient, the electroporation ablation system comprising:
an ablation catheter including:
a handle;
a shaft having a distal end; and
catheter electrodes situated at the distal end of the shaft and spatially arranged to generate electric fields in the targeted tissue in response to electrical pulses; and
an electroporation generator operatively coupled to the catheter electrodes and configured to deliver the electrical pulses in an electroporation pulse sequence to one or more catheter electrodes,
wherein the electroporation pulse sequence includes multiple pulse bursts applied across multiple heart beats, one pulse burst per heartbeat, each of the multiple pulse bursts including biphasic pulses separated by an inter-pulse length of between 200 and 350 microseconds to provide reduce muscle stimulation while creating irreversible electroporation lesions.

14. The electroporation ablation system of claim 13, wherein each pulse burst of the multiple pulse bursts is gated to an R-wave in the heartbeat and applied during a ventricle refractory period of the heartbeat.

15. The electroporation ablation system of claim 13, wherein each of the biphasic pulses includes a positive pulse portion and a negative pulse portion, and each of the positive pulse portion and the negative pulse portion has a pulse width of between 1 and 5 microseconds.

16. The electroporation ablation system of claim 13, wherein each of the biphasic pulses includes a positive pulse portion and a negative pulse portion with an inter-phase delay between the positive pulse portion and the negative pulse portion of between 0 and 10 microseconds.

17. A method of ablating targeted tissue in a patient by irreversible electroporation, the method comprising:
delivering an irreversible electroporation pulse sequence including:
delivering multiple pulse bursts across multiple heart beats, each of the multiple pulse bursts including biphasic pulses separated by an inter-pulse length of between 200 and 350 microseconds to reduce muscle stimulation while creating irreversible electroporation lesions.

18. The method of claim 17, wherein delivering multiple pulse bursts across multiple heart beats includes delivering the biphasic pulses that each have a positive pulse portion and a negative pulse portion each having a pulse width of between 1 and 5 microseconds.

19. The method of claim 17, wherein delivering multiple pulse bursts across multiple heart beats includes delivering the biphasic pulses that each have a positive pulse portion and a negative pulse portion separated by an inter-phase delay of between 0 and 10 microseconds.

20. The method of claim 17, wherein delivering multiple pulse bursts across multiple heart beats includes delivering one pulse burst per heartbeat.
